(19) United States
(12) Patent Application Publication
(10) Pub. No.: US 2019/0095635 A1
(43) Pub. Date: Mar. 28, 2019

(54) METHOD AND DEVICE FOR MANAGING SYSTEM ACCESS TO A USER

(71) Applicant: Wipro Limited, Bangalore (IN)

(72) Inventors: Venkata Subramanian Jayaraman, Chennai (IN); Sumithra Sunderasan, Chennai (IN)

(73) Assignee: Wipro Limited

(21) Appl. No.: 15/819,010

(22) Filed: Nov. 21, 2017

(30) Foreign Application Priority Data
Sep. 28, 2017 (IN) 201741034472

(51) Int. Cl. G06F 21/62; G16H 50/30

(52) U.S. Cl. CPC G06F 21/6218; G16H 50/30

(57) ABSTRACT

A method and device are described for managing system access to a user. The method includes receiving at least one of user-profile parameters, work-profile parameters and health parameters associated with the user. The method includes determining, one or more patterns from the at least one of user-profile parameters, work-profile parameters and health parameters associated with the user. The method further includes determining a stress level of the user based on the one or more patterns. The method further includes, managing access to at least one of one or more devices, user accounts, or user access cards associated with the user based on the stress level.

### TECHNICAL FIELD

The present subject matter is related, in general, to managing system accessibility and more particularly, but not exclusively, to a method and device for managing system access to a user based on a stress level of the user.

### BACKGROUND

High stress of workers in white-collar jobs are talking a toll on the health of employees and also affecting productivity of the employees. This is specially an issue in job sectors like pharmacy and medical counselling where there is direct interaction with clients. Stress in such work may directly impact the output of the work, affect the interaction with the clients and as a result affect the performance of the employees as well as reputation of the organization. Adding to the problem is the fact that most of the stressed workers may not agree or even know that they are stressed. Thus, the employees may not take adequate breaks when required. Dangerous long term effects due to work stress may be avoided if break time is offered at correct time intervals. The present systems may act only on information that is provided by the user and may not be able to intelligently provide system access to the employees based on their health condition.

Further limitations and disadvantages of conventional and traditional approaches will become apparent to one of skill in the art, through comparison of described systems with some aspects of the present disclosure, as set forth in the remainder of the present application and with reference to the drawings.

### SUMMARY

According to embodiments illustrated herein, a method for managing system access to a user is disclosed. The method may include, receiving, by an access management device, at least one of user-profile parameters, work-profile parameters and health parameters associated with the user. The method may include determining, by the access management device, one or more patterns from at least one of the user-profile parameters, the work-profile parameters and the health parameters associated with the user. The method may include determining, by the access management device, a stress level of the user based on the one or more patterns. The method may further include managing, by the access management device, system access based on the stress level.

According to embodiments illustrated herein, an access management device for managing system access to a user is disclosed. The access management device may comprise a processor and a memory communicatively coupled to the processor. The memory may store instructions which may cause the processor to receive at least one of user-profile parameters, work-profile parameters and health parameters associated with the user. The memory may store instructions which may cause the processor to determine one or more patterns from at least one of the user-profile parameters, the work-profile parameters and the health parameters associated with the user. The memory may further store instructions to determine a stress level of the user based on the one or more patterns. The memory may also store instructions to manage system access based on the stress level.

Disclosed herein is a non-transitory computer-readable storage medium having stored thereon, a set of computer-executable instructions for causing a computer comprising one or more processors to perform steps of managing system access to a user. The computer-executable instructions may include instructions to receive at least one of user-profile parameters, work-profile parameters and health parameters associated with the user. The computer-executable instructions may also include instruction to determine one or more patterns from at least one of the user-profile parameters, the work-profile parameters and the health parameters associated with the user. The computer-executable instructions may further include instructions to determine a stress level of the user based on the one or more patterns and manage system access based on the stress level.

The foregoing summary is illustrative only and not intended to be limiting in any way. In addition to the illustrative aspects, embodiments, and features described above, further aspects, embodiments, and features will become apparent by reference to the drawings and the following

### BRIEF DESCRIPTION OF THE ACCOMPANYING DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of device and/or system and/or methods in accordance with embodiments of the present subject matter are now described, by way of example only, and with reference to the accompanying figures.

FIG. 1 illustrates an exemplary system environment in which various embodiments of the present disclosure may function.

FIG. 2 is a block diagram illustrating an access management device for managing system access to a user in accordance with some embodiments of the present disclosure.

FIG. 3 is a flowchart illustrating a method of managing system access to a user in accordance with some embodiments of the present disclosure.

FIG. 4 is a Hock diagram illustrating an exemplary computer system for implementing embodiments consistent with the present disclosure.

It should be appreciated by those skilled in the art that any block diagrams herein represent conceptual views of illustrative systems or devices embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

The present disclosure may be best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods, systems and devices may extend beyond the described embodiments. For example, the teachings presented and the needs of a particular application may yield multiple alternative and suitable approaches to implement the functionality of any detail described herein. Therefore, any approach may extend beyond the particular implementation choices in the following embodiments described and shown.

References to “one embodiment,” “at least one embodiment,” “an embodiment,” “one example,” “an example,” “for example,” and the like indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” or “in some embodiments ”does not necessarily refer to the same embodiment.

The present invention discloses an access management method and device that enables managing system access to a user based on the stress level of the user. In some embodiments, managing system access to users may refer to activating or deactivating one or more user devices that may be accessed by the user in real time. Further, managing system access may include activating or deactivating user accounts or user access cards temporarily or permanently based on the stress level of the user.

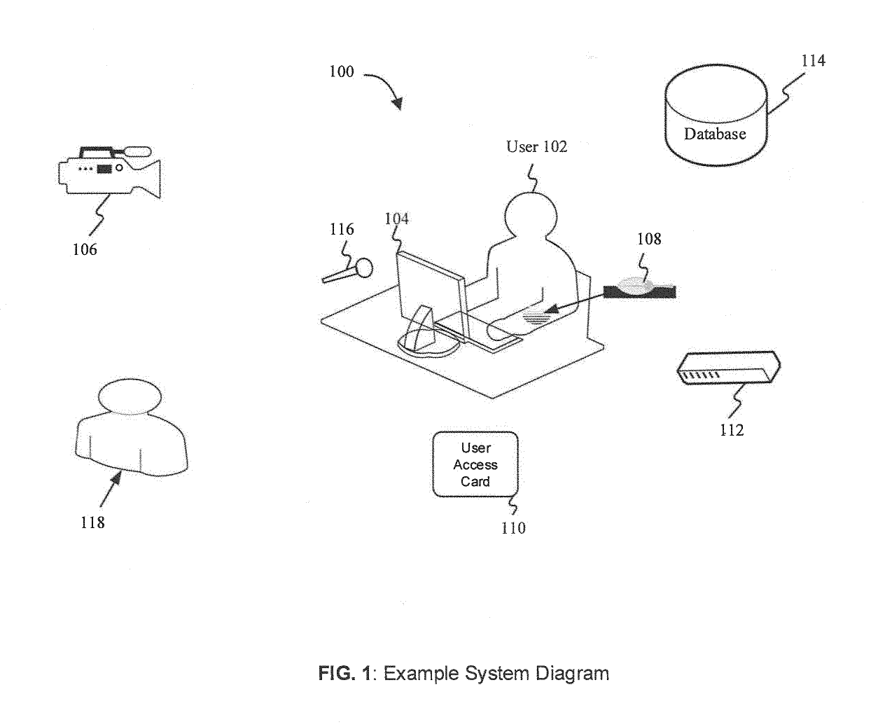

FIG. 1 illustrates an exemplary environment 100 in which various embodiments of the present disclosure may function. The environment 100 may include a user 102 using user computer 104. The user 102 may have an access card 110, and wear a wearable device 108, which may be able to measure heart rate and blood pressure of the user. It may be noted that in some embodiments, the blood pressure monitoring device and the heart rate monitoring device may be two separate devices. In some embodiments, the wearable device 108 may be a fitness tracking device or any portable health-monitoring device that can monitor heart rate and blood pressure of the user.

The environment may include one or more image sensors such as camera 106, which may perform facial recognition of the user to determine facial expressions of the user. The environment 100 may include a microphone 116 to detect voice tone from voice of the user 102. Both facial expression and voice tone may be used to determine stress level of the user 102. The system 100 has an access management device 112 that may be communicatively coupled to the user computer 104, database 114, camera 106, user access card 110 and microphone 116.

In accordance with some embodiments, communication between the access management device 112 and user computer 104, database 114, camera 106, user access card 110, and microphone 116 and various other connected devices maybe performed, in accordance with various wired and wireless communication protocols. Examples of such wired and wireless communication protocols include, but are not limited to, Transmission Control Protocol and Internet Protocol (TCP/IP), User Datagram Protocol (UDP), Hypertext Transfer Protocol (HTTP), File Transfer Protocol (FTP), ZigBee, EDGE, infrared (IR), IEEE 802.11, 802.16, 2G, 3G, 4G cellular communication protocols, and/or Bluetooth (BT) communication protocols. The communication network may include, but is not limited to, the Internet, a cloud network, a Wireless Fidelity (Wi-Fi) network, a Wireless Local Area Network (WLAN), a Local Area Network (LAN), a telephone line (POTS), and/or a Metropolitan Area Network (MAN).

The access management device 112 may receive as inputs, user-profile parameters from the user computer 104 or the database 114. The user-profile parameters may include information such as name, age, sex and blood group of the user. The access management device 112 may also receive health parameters associated with the user in real time from the camera 106, microphone 116 and the wearable device 108. In some embodiments, the health parameters of the user may correspond to real time health parameters and user's health history. The health history of the user 102 may be fetched from an external source such as the database 114 or from any device associated with the user 102 such as the user computer 104 or a portable computing device such as mobile, laptop tablet and the like (not shown in FIG. 1). The user's real time health parameters are crucial for managing system accessibility for the user. The real time health parameters may include heart rate, blood pressure, health history, speech quality and facial expression of the user. The camera 106 may record the user's facial expression and provide image input to the access management device 112. The microphone 116 may provide voice input of the user to the access management device 112.

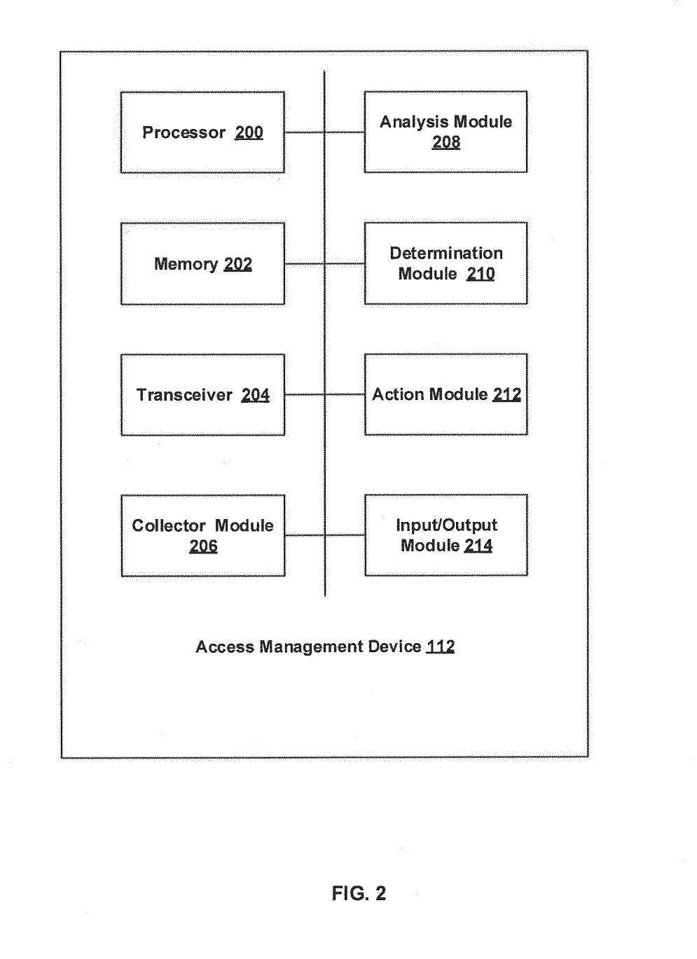

In accordance with some embodiments of the invention, the access management device 112 may receive images as inputs from one or more image-sensing devices such as camera 106. Further, the access management device 112 may receive audio inputs from one or more microphones such as microphone 116. In some embodiments, the images may be of the user while the user interacts with client 118. In some embodiments, the user 102 may be a pharmacist and the client 118 may be a patient that the pharmacist is counselling. In some embodiments, the images may be primarily of the user's face during speech or during user's interaction with the client 118. In addition to the user-profile parameters and the health parameters, the access management device 112 may also receive work-profile parameters associated with the user. The user -profile parameters, the health parameters and the work-profile parameters may be used by the access management device to determine stress level of the user. Determining stress based on these parameters is further explained in conjunction with FIG. 2. The work-profile parameters may include at least one of average number of human interactions per hour, work shift time, total number of clients served, continuously worked hours, work experience, and total working days in a week.

Referring now to FIG. 2, a block diagram of various components within the access management device 112 is illustrated, in accordance with an embodiment of the present disclosure. The access management device 112 may be implemented as a variety of portable computing devices, such as, a smartphone, a dedicated handheld device, a tablet, or a phablet. Access management device 112 may be also integrated to the user computer 104. Alternatively, the access management device 112 may be implemented as a distributed system, such that, few components of the access management device 112 reside in a portable device, while, other components may reside in a server, which may be located remotely or integrated to the user computer 104.

The access management device 112 may include a processor 200, a memory 202, transceiver 204, collector module 206, analysis module 208, determination module 210, action module 212 and input/output module 214. Each of the components and modules maybe communicatively coupled to each other. It will be appreciated that the above components may be represented as a single module/component or a combination of different modules/components.

The processor 200 comprises suitable logic, circuitry, and interfaces that may be configured to execute a set of instructions stored in the memory 202. The processor 200 may be implemented based on a number of processor technologies known in the art. Examples of the processor 200 include, but are not limited to, an X86-based processor, a Reduced instruction Set Computing (RISC) processor, an Application-Specific Integrated Circuit (ASIC) processor, a Complex Instruction Set Computing (CIBC) processor, and/or other processor.

The memory 202 has processor instructions stored thereon, which on execution cause processor 200 to manage system access to a user. The memory 202 may be configured to store one or more programs, routines, or scripts that may be executed in coordination with the processor 200. In some embodiments, the memory 202 includes instructions stored thereon that on execution cause the processor 200 to activate or deactivate, devices used by the user, user accounts, or user access cards. The memory 202 may be implemented based on a Random Access Memory (RAM), a Read-Only Memory (ROM), a Hard Disk Drive (HDD), a storage server, and/or a Secure Digital (SD) card. The memory 202 may be a non-volatile memory or a volatile memory. Examples of non-volatile memory, may include, but are not limited to a flash memory, a Read Only Memory (ROM), a Programmable ROM (PROM), Erasable PROM (EPROM), and Electrically EPROM (EEPROM) memory. Examples of volatile memory may include, but are not limited Dynamic Random Access Memory (DRAM), and Static Random-Access memory (SRAM).

The access management device 112 may include a transceiver 204 as shown in FIG. 2 to retrieve data from various sources. The transceiver 204comprises suitable logic, circuitry, and interfaces that may be configured to retrieve data pertaining to health history of the user from external sources such as database 114. The transceiver may alternatively transmit information from the access management device 112 to other devices communicatively coupled to the access management device 112. The transceiver 204 may also transmit control information from the access management device 112 to enable activation or deactivation of one or more devices associated with the user 102. The transceiver 204 may implement one or more known technologies to support wired or wireless communication with the communication network. In an embodiment, the transceiver 204 may include, but is not limited to, an antenna, a radio frequency (RF) transceiver, one or more amplifiers, a tuner, one or more oscillators, a digital signal processor, a Universal Serial Bus (USB) device, a coder-decoder (CODEC) chipset, a subscriber identity module (SIM) card, and/or a local buffer.

The transceiver 204 may communicate via wireless communication with networks, such as the Internet, an Intranet and/or a wireless network, such as a cellular telephone network, a wireless local area network (LAN) and/or a metropolitan area network (MAN). The wireless communication may use any of a plurality of communication standards, protocols and technologies, such as: Global System for Mobile Communications (GSM), Enhanced Data GSM Environment (EDGE), wideband code division multiple access (W-CDMA), code division multiple access (CDMA), time division multiple access (TDMA), Bluetooth, Wireless Fidelity (e,g., IEEE. 802.11a, IEEE 802.11b, IEEE 802.11g and/or IEEE 802.11n), voice over Internet Protocol (VoIP), WI-MAX, a protocol for email, instant messaging, and/or Short Text message Service (SMS).

The access management device 112 may include a collector module 206 that may collect one or more parameters associated with the user. The collector module 206 may receive profile parameters, work-profile parameters and health parameters associated with the user. The user-profile parameters may include name, age, sex and blood group of the user. The work-profile parameters associated with the user may include at least one of average number of human interactions per hour, work shift time, total number of clients served, continuously worked hours, work experience, and total working days in a week. The collector module 206 may further collect the health parameters associated with the user which may include at least one of heart rate, blood pressure, health history, speech quality and facial expression of the user. The user-profile parameters, work-profile parameters and health parameters associated with the user may be criteria for determining stress level of the user.

The access management device 112 may include an analysis module 208 that may be configured to derive patterns from the various parameters collected over a period of time. The analysis module 208 may correlate various parameters to determine patterns. For example, the analysis module may determine that a user who has more experience is generally more relaxed. This correlation could be made from comparing the user-profile parameters with various health parameters. When the heart rate or the pulse rate of the user remains more or less constant irrespective of the number of clients handled by the user, then it may be inferred that since the user is experienced, the user is able to handle more interactions without being stressed. The reverse may also be true as a user with lower experience may not fully understand how to communicate or handle a potentially difficult client which may in turn lead to the user being stressed.

In another example, the analysis module 208 may correlate the speech quality (or the facial expressions) of the user with the blood pressure to infer that the user might be stressed. If the user sounds tensed or provocative and the health parameters indicate that the user's blood pressure is high, then the analysis module 208 may conclude that the user is stressed. Similarly, if the user's facial expressions indicate anger or frustration, then the analysis module 208 may conclude that the user is stressed. In some embodiments, a sample of the user's speech in normal or unstressed condition may be recorded and kept in the memory for the determination of stress. The individual speech of the user 102 may be analyzed against the sample of voice tone to determine stress. In some embodiments, the speech or voice tone may determine the stress level of the user. In some embodiments, user's emotions may be detected from the facial expressions and may be used to detect stress level of the user.

It will be apparent to a person skilled in the art that various correlations may be made between the work-profile parameters, the health parameters and the user-profile parameters to determine stress without deviating from the scope of the present disclosure.

In some embodiments, patterns relating to the load of the user may be determined by the analysis module 208. The load of the user may be determined based on the number of interactions with clients per hour, work shift time, total number of clients served, etc. The analysis module 208 may determine patterns by correlating the load of the user to the stress caused. For example, the analysis module 208 may determine that when the user has a work shift time greater than seven hours, the user's blood pressure generally goes beyond normal limits. This may cause analysis module 208 to infer that if the user works for more than seven hours at a time, the user may have a certain level of stress. In some embodiments, the correlation of the various parameters and their impact on the stress of the user may be saved and a historic profile may be created.

Under the current load of the user, the historic patterns may be used to determine if the user has a propensity towards being stressed. In this way, the analysis module 208 determines patterns between the work parameters, the user parameters and the health parameters that may lead to stressful conditions for the user. Further, the analysis module 208 may compare the various parameters associated with the user with corresponding threshold values and determine if a particular parameter is out of normal bounds. For example, the analysis module 208 may assign a value ‘Normal’ if the heart rate of the user is between 60-72 beats per minute. Likewise, the heart rate may be considered ‘High’ if the beats per minute is above 100. The heart rate may be considered ‘Medium’ if it lies between ‘High’ and ‘Normal’. In addition to comparing individual parameters with corresponding threshold values, the total number of parameters outside of normal ranges may indicate if the user is “Highly Stressed” or “Normal” or “Moderately Stressed”. For example, if the health parameters indicate that user shows a high blood pressure and the speech quality indicates the user is tensed, and the facial expression shows reddening of the face, then the analysis module 208 may determine that the user is “Highly stressed”. Table 1 denotes exemplary parameter values and corresponding levels of stress.

TABLE 1

| | Health | Speech | Facial | |
|---|---|---|---|---|
| User's Name | Condition | Quality | Expression | Stress Level |
| Professional X | High BP | Tensed | Reddening | Highly |
| | | | of face | stressed |
| Professional Y | Normal | Normal | Normal | Normal |
| Professional Z | Diabetes | Feeble | Pleasant | Normal |
| Professional V | NA | Tensed | Raised | Moderately |
| | | | Eyebrow | stressed |
| Professional K | NA | Louder than | Tensed | Moderately |
| | | normal | | stressed |

On determining a stress level of the user, the access management device 112 may either provide recommendations to the user or automatically take one or more actions. In some embodiments, the access management device 112 may recommend a break to the user so as to bring down the stress level of the user. In other embodiments, the access management device 112 may determine stress level of the user and freeze one or more accounts or access cards associated with the user if the stress level is determined to be beyond a predefined threshold level. In some embodiments, the access management device 112 may continuously monitor the various parameters and enable access to devices or accounts or access cards only when the stress level of the user comes down below a predefined threshold level. Exemplary recommendations provided to the user based on stress level of the user is illustrated in Table 2.

TABLE 1

| User | Stress Level | Recommendation |
|---|---|---|
| Professional X | Highly stressed | Immediate Break |
| Professional Y | Normal | No Break |
| Professional Z | Normal | No Break |
| Professional V | Moderately stressed | Break after an hour |
| Professional K | Highly stressed | Immediate Break |

The determination module 210 may determine action based on the stress level of the user 102. The action module 212 may get input about each user from the determination module 210 and trigger corresponding actions based on the stress level to the one or more user-devices, user-profiles. In some embodiments, the action module 212 may provide recommendations to the user 102 or to a higher authority. In the above example since professional X has been determined to be under high stress the determination module 210of the access management device 112 may block all access to the user devices, user profiles and user's access card to any working systems.

In some embodiments the access management device 112 may force the user 102 like professional X to take a break. In some embodiments the access management system 112 may continuously monitor the blood pressure and the heart rate of the professional X to determine the time period till the system access may be blocked for the professional X. Professional K may be subject to similar access blocks to the pharmacy system. Once the user is detected to be again in low stress level the access may be provided by the access management device 112. Likewise, from example in Table 1, Professional Y, Z and V may not be provided break for work immediately. However Professional V may be recommended for taking a break and there may be partial blocking access for Professional V. For example, the user computer may not allow any more data entry for counselling of clients when the stress level of professional V is detected to be “moderately stressed”. Alternatively, billing may be blocked or generation of prescription may be disabled. Access to the user's profile may be blocked after an hour for a predetermined period after an initial alert.

Further, the input/output module 214 may display the analytic data and access blocking information in one or more devices associated with the user such as user computer 104 and user mobile device. In some embodiments, the input/output module 214 may display the user health history and the detected stress level of the user in the display of the access management device 112. In some embodiments, the input/output module may be configured to take inputs from the user.

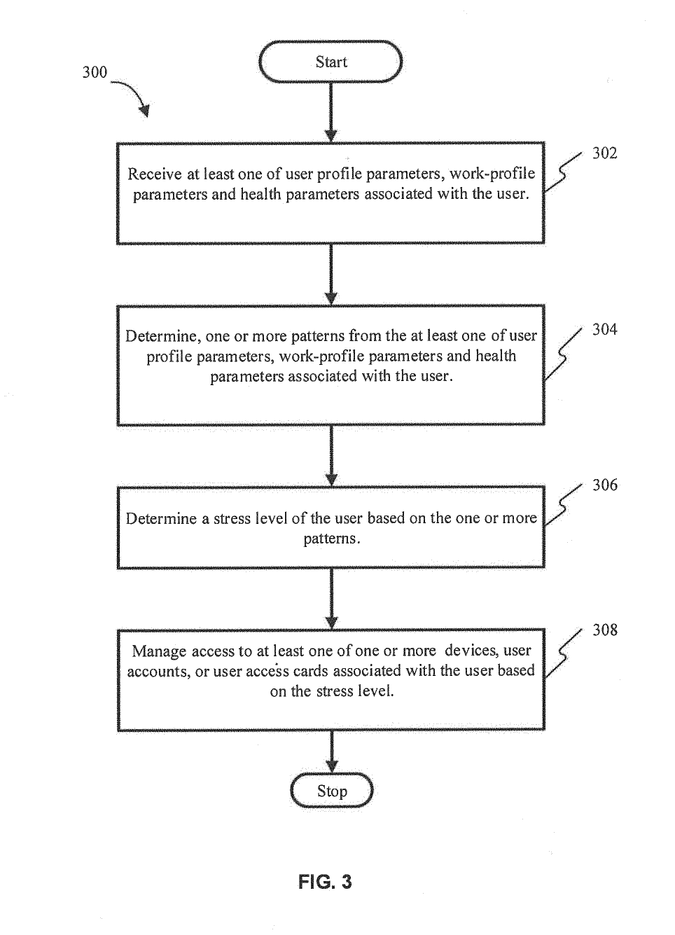

FIG. 3 shows a flowchart illustrating for managing system access to a user, in accordance with some embodiments of the present disclosure. As illustrated in FIG. 3, the method 300 includes one or more blocks illustrating a method of managing system access to a user, by the access management device 112. The method 300 may be described in the general context of computer executable instructions. Generally, computer executable instructions can include routines, programs, objects, components, data structures, procedures, modules, and functions, which perform functions or implement abstract data types. The order in which the method 300 is described is not intended to be construed as a limitation, and any number of the described method blocks can be combined in any order to implement the method 300. Additionally, individual blocks may be deleted from the methods without departing from the spirit and scope of the subject matter described herein. Furthermore, the method 300 can be implemented in any suitable hardware, software, firmware, or combination thereof.

At step 302, the method 300 may include receiving, by the access management device 112, at least one of user-profile parameters, workprofile parameters and health parameters associated with the user. In some embodiments, the user-profile parameters include name, age, sex and blood group of the user. In some embodiments, the work-profile parameters associated with the user may include at least one of average number of human interactions per hour, work shift time, total number of clients served, continuously worked hours, work experience, and total working days in a week. In some embodiments, the health parameters associated with the user may include at least one of heart rate, blood pressure, health history, speech quality and facial expression of the user.

At block 304, the method may include determining, by the access management device, one or more patterns from the at least one of user-profile parameters, work-profile parameters and health parameters associated with the user. Further, at block 306, the method may include determining, by the access management device, a stress level of the user based on the one or more patterns. In some embodiments, load of the user may be determined based on at least one of the user-profile parameters, the work-profile parameters and the health parameters associated with the user. The stress level of the user may be determined based on the load of the user. Determination of stress level of the user have been explained in details in conjunction with FIG. 2.

At block 308, the method may include managing access to at least one of one or more devices, user accounts, or user access cards associated with the user based on the stress level. In some embodiments, managing the at least one of one or more devices, user accounts, or user access cards may include at least one of activating and deactivating the at least one of one or more devices, user accounts, or user access cards. In an example, if the detected stress level is high the user's access card may be disabled to prevent the user from entering a room such as an operation theatre or patient counselling room if the user is a doctor or a medical counsellor. In another example if the stress level of the user 102 is medium, then the user may be allowed to counsel clients who may be patients, however the user's access to an operation theatre may be blocked.

The illustrated operations of FIG. 3 and show certain events occurring in a certain order. In alternative embodiments, certain operations may be performed in a different order, modified or removed. Moreover, steps may be added to the above described logic and still conform to the described embodiments. Further, operations described herein may occur sequentially or certain operations may be processed in parallel. Yet further, operations may be performed by a single processing unit or by distributed processing units.

### Computer System

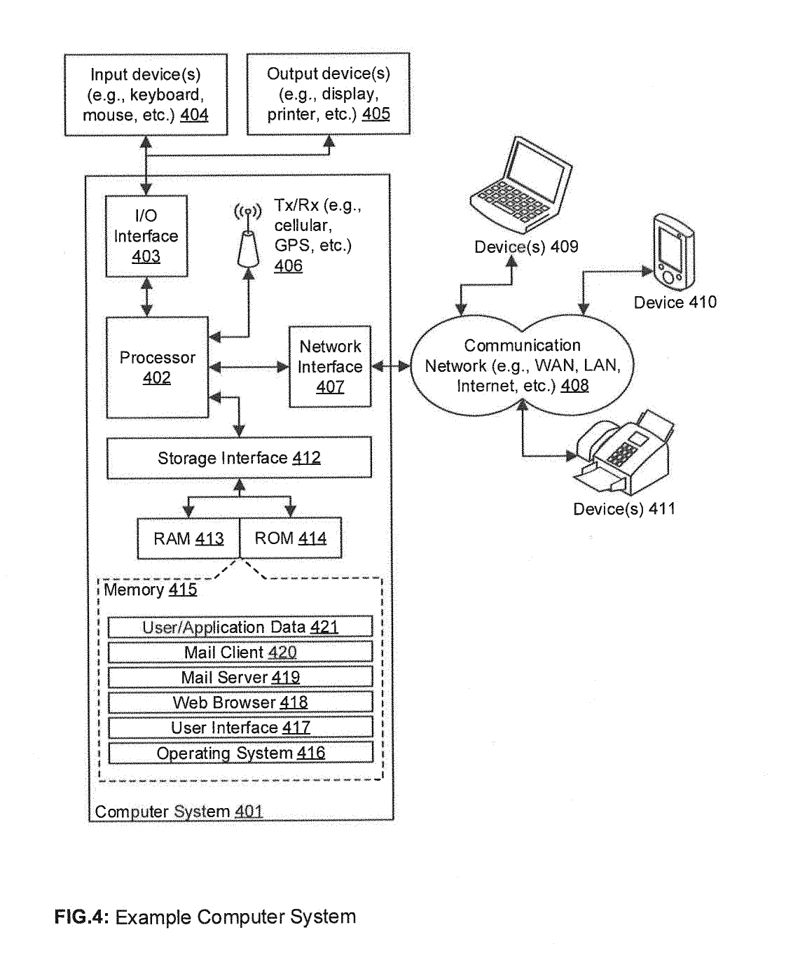

FIG. 4 illustrates a block diagram of an exemplary computer system for implementing embodiments consistent with the present disclosure. Variations of computer system 401 may be used for managing system access to a user. The computer system 401 may comprise a central processing unit (“CPU” or “processor”) 402. The processor 402 may comprise at least one data processor for executing program components for executing user-generated or system-generated requests. A user may include a person, a person using a device such as those included in this disclosure, or such a device itself. The processor 402 may include specialized processing units such as integrated system (bus) controllers, memory management control units, floating point units, graphics processing units, digital signal processing units, etc. The processor may include a microprocessor, such as AMD Athlon, Duron or Opteron, AMA's application, embedded or secure processors, IBM PowerPC, Intel's Core, Itanium, Xeon, Celeron or other line of processors, etc. The processor 402 may be implemented using mainframe, distributed processor, multicore, parallel, grid, or other architectures. Some embodiments may utilize embedded technologies like application-specific integrated circuits (ASICs), digital signal processors (DSPs), Field Programmable Gate Arrays (FPGAs), etc.

The processor 402 may be disposed in communication with one or more input/output (I/O) devices via I/O interface 403. The I/O interface 403 may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/n/x, Bluetooth, cellular (e.g., code-division multiple access (CDMA), high-speed packet access (HSPA+), global system for mobile communications (OSM), long-term evolution (LTE), WiMax, or the like), etc.

Using the I/O interface 403, the computer system 401 may communicate with one or more I/O devices. For example, the input device 404 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, etc. Output device 405 may be a printer, fax machine, video display (e.g., cathode ray tube (CRT), liquid crystal display (LCD), light-emitting diode (LED), plasma, or the like), audio speaker, etc. In some embodiments, a transceiver 406 may be disposed in connection with the processor 402. The transceiver may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G HSDPA/HSUPA communications, etc.

In some embodiments, the processor 402 may be disposed in communication with a communication network 408 via a network interface 407. The network interface 407 may communicate with the communication network 408. The network interface 407 may employ connection protocols including, without limitation, direct connect, Ethernet (e.g., twisted pair 10/100/1000 Base T), transmission control protocol/internet protocol (TCP/IP), token ring, IEEE 802,11a,/b/g/n/x, etc. The communication network 408 may include, without limitation, a direct interconnection, local area network (LAN), wide area network (WAN), wireless network (e.g., using Wireless Application Protocol), the Internet, etc. Using the network interface 407 and the communication network 408, the computer system 401 may communicate with devices 410, 411, and 412. These devices may include, without limitation, personal computer(s), server(s), fax machines, printers, scanners, various mobile devices such as cellular telephones, smartphones (e.g., Apple iPhone, Blackberry, Android-based phones, etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, the computer system 401 may itself embody one or more of these devices.

In some embodiments, the processor 402 may be disposed in communication with one or more memory devices referred in the FIG. 4 as Memory 415 (e.g., RAM 413, ROM 414, etc.) via a storage interface 412. The storage interface 802 may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, solid-state drives, etc.

The memory devices may store a collection of program or database components, including, without limitation, an operating system 416, user interface application 417, web browser 418, mail server 419, mail client 420, user/application data 421 (e.g., any data variables or data records discussed in this disclosure), etc. The operating system 416 may facilitate resource management and operation of the computer system 401. Examples of operating systems include, without limitation, Apple Macintosh OS X, UNIX, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like. User interface 417 may facilitate display, execution, interaction, manipulation, or operation of program components through textual or graphical facilities. For example, user interfaces may provide computer interaction interface elements on a display system operatively connected to the computer system 401, such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, Javascript, AJAX, HTML, Adobe Flash, etc.), or the like.

In some embodiments, the computer system 401 may implement a web browser 418 stored program component. The web browser 418 may be a hypertext viewing application, such as Microsoft Internet Explorer, Google Chrome, Mozilla Firefox, Apple Safari, etc. Secure web browsing may be provided using HTTPS (secure hypertext transport protocol), secure sockets layer (SSW, Transport Layer Security (TLS), etc. Web browsers may utilize facilities such as AJAX, DHTML, Adobe Flash, JavaScript, Java, application programming interfaces (APIs), etc. In some embodiments, the computer system 401 may implement a mail server 419 stored program component. The mail server 419 may be an Internet mail server such as Microsoft Exchange, or the like. The mail server may utilize facilities such as ASP, ActiveX, ANSI C++/C#, Microsoft .NET, CCI scripts, Java, JavaScript, PERL, PHP, Python, WebObjects, etc. The mail server 419 may utilize communication protocols such as Internet text message access protocol (IMAP), messaging application programming interface (MAPI), Microsoft Exchange, post office protocol (POP), simple mail transfer protocol (SMTP), or the like. In some embodiments, the computer system 401 may implement a mail client 420 stored program component. The mail client 420 may be a mail viewing application, such as Apple Mail, Microsoft Entourage, Microsoft Outlook, Mozilla Thunderbird, etc.

In some embodiments, computer system 401 may store user/application data 421, such as the data, variables, records, etc. as described in this disclosure. Such databases may be implemented as fault-tolerant, relational, scalable, secure databases such as Oracle or Sybase. Alternatively, such databases may be implemented using standardized data structures, such as an array, hash, linked list, structured text file (e.g., XML), table, or as object-oriented databases (e.g., using ObjectStore, Poet, Nope, etc.). Such databases may be consolidated or distributed, sometimes among the various computer systems discussed above in this disclosure. It is to be understood that the structure and operation of the any computer or database component may be combined, consolidated, or distributed in any working combination.

The specification has described method and device for controlling an autonomous vehicle using location based dynamic dictionary. The illustrated steps are set out to explain the exemplary embodiments shown, and it should be anticipated that ongoing technological development will change the manner in which particular functions are performed. These examples presented herein are for purposes of illustration, and not limitation. Further, the boundaries of the functional building blocks or modules have been arbitrarily defined herein for the convenience of the description. Alternative boundaries or modules can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

The present invention is based on managing system access to an user. Most of these advantages flow from the fact that an user may be automatically barred from accessing devices and profiles that would in turn disable the user from working when the user is in stressed condition. The system disclosed automatically improves quality of work and reduces health hazards by preventive measures.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise. The terms “including”, “comprising”, “having” and variations thereof mean “including but not limited to”, unless expressly specified otherwise. The terms “a”, “an” and “the” mean “one or more”, unless expressly specified otherwise.

A description of an embodiment with several components or modules in communication with each other does not imply that all such components are required. On the contrary, a variety of optional components or modules are described to illustrate the wide variety of possible embodiments of the invention.

The language used in the specification has been principally selected for readability and instructional purposes, and it may not have been selected to delineate or circumscribe the inventive subject matter. It is therefore intended, that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted for carrying out the methods described herein may be suited. A combination of hardware and software may be a general-purpose computer system with a computer program that, when loaded and executed, may control the computer system such that it carries out the methods described herein. The present disclosure may be realized in hardware that comprises a portion of an integrated circuit that also performs other functions.

A person with ordinary skills in the art will appreciate that the devices, systems, modules, and sub-modules have been illustrated and explained to serve as examples and should not be considered limiting in any manner. It will be further appreciated that the variants of the above disclosed device elements, system elements, modules, and other features and functions, or alternatives thereof, may be combined to create other different devices, systems or applications.

Those skilled in the art will appreciate that any of the aforementioned steps, device modules and/or system modules may be suitably replaced, reordered, or removed, and additional steps, device modules and/or system modules may be inserted, depending on the needs of a particular application. In addition, the system or devices of the aforementioned embodiments may be implemented using a wide variety of suitable processes and system modules, and are not limited to any particular computer hardware, software, middleware, firmware, microcode, and the like. The claims can encompass embodiments for hardware and software, or a combination thereof.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

We claim:

1) A method of managing system access to a user, the method comprising:
receiving, by an access management device, at least one of user-profile parameters, work-profile parameters and health parameters associated with the user;
determining, by the access management device, one or more patterns from at least one of the user-profile parameters, the work-profile parameters and the health parameters associated with the user;
determining, by the access management device, a stress level of the user based on the one or more patterns; and
managing, by the access management device, system access based on the stress level.

2) The method as claimed in claim 1, wherein managing system access comprises at least one of activating and deactivating at least one of one or more devices, user accounts, or user access cards.

3) The method as claimed in claim 1, wherein the work-profile parameters associated with the user comprise at least one of average number of human interactions per hour, work shift time, total number of clients served, continuously worked hours, work experience, and total working days in a week.

4) The method as claimed in claim 1, wherein the health parameters associated with the user comprise at least one of heart rate, blood pressure, health history, speech quality and facial expression of the user.

5) The method as claimed in claim 1, wherein the user-profile parameters comprise name, age, sex and blood group of the user.

6) The method as claimed in claim 1 further comprising, providing recommendation based on the stress level of the user.

7) The method as claimed in claim 1 further comprising monitoring stress level of the user to enable system access.

8) The method as claimed in claim 1, further comprising determining load of the user based on at least one of the user-profile parameters, the work-profile parameters and the health parameters associated with the user.

9) An access management device for managing system access to a user, the access management device comprising:
a processor; and
a memory communicatively coupled to the processor, wherein the memory stores processor instructions, which, on execution, causes the processor to:
receive at least one of user-profile parameters, work-profile parameters and health parameters associated with the user;
determine one or more patterns from the at least one of user-profile parameters, work-profile parameters and health parameters associated with the user;
determine a stress level of the user based on the one or more patterns; and
manages system access based on the stress level.

10) The device as claimed in claim 9, wherein managing system access comprises at least one of activating and deactivating at least one of one or more devices, user accounts, or user access cards.

11) The device as claimed in claim 9, wherein the work-profile parameters associated with the user comprise at least one of average number of human interactions per hour, work shift time, total number of clients served, continuously worked hours, work experience, and total working days in a week.

12) The device as claimed in claim 9, wherein the health parameters associated with the user comprise at least one of heart rate, blood pressure, health history, speech quality and facial expression of the user.

13) The device as claimed in claim 9, wherein the user-profile parameters comprise name, age, sex and blood group of the user.

14) The device as claimed in claim 9, further comprising, providing recommendations based on the stress level of the user.

15) The device as claimed in claim 9, further comprising, monitoring stress level of the user to enable system access.

16) The device as claimed in claim 9, further comprising determining load of the user based on at least one of the user-profile parameters, the work-profile parameters and the health parameters associated with the user.

17) A non-transitory computer-readable storage medium having stored thereon, a set of computer-executable instructions for causing a computer comprising one or more processors to perform steps comprising:
receiving at least one of user-profile parameters, work-profile parameters and health parameters associated with the user;
determining one or more patterns from the at least one of the user-profile parameters, the work-profile parameters and the health parameters associated with the user;
determining a stress level of the user based on the one or more patterns; and
managing system access based on the stress level.

18) The non-transitory computer-readable storage medium as claimed in claim 17, wherein the work-profile parameters associated with the user comprise at least one of average number of human interactions per hour, work shift time, total number of clients served, continuously worked hours, work experience, and total working days in a week.

19) The non-transitory computer-readable storage medium as claimed in claim 17, wherein the health parameters associated with the user comprise at least one of heart rate, blood pressure, health history, speech quality and facial expression of the user.

20) The non-transitory computer-readable storage medium as claimed in claim 17, wherein the user-profile parameters comprise name, age, sex and blood group of the user.
